(19) United States
(12) Patent Application Publication
(10) Pub. No.: US 2022/0229048 A1
(43) Pub. Date: Jul. 21, 2022

(54) HIGH-BRIGHTNESS FLUOROPHORES BY COVALENT FUNCTIONALIZATION

(71) Applicant: Michigan Technological University, Houghton, MI (US)

(72) Inventors: Yoke Khin Yap, Houghton, MI (US); Dongyan Zhang, Houghton, MI (US); Rodney Oakley, Hancock, MI (US); Nazmiye Yapici, South Lyon, MI (US); Xiuling Liu, Ann Arbor, MI (US)

(21) Appl. No.: 17/615,441

(22) PCT Filed: Jun. 1, 2020
(86) PCT No.: PCT/US2020/035574

§ 371 Date: Nov. 30, 2021

Related U.S. Application Data
(63) Continuation-in-part of application No. 15/953,200, filed on Apr. 13, 2018.
(60) Provisional application No. 62/485,379, filed on Apr. 13, 2017.
(60) Provisional application No. 62/855,128, filed on May 31, 2019.

(51) Int. Cl. G01N 33/533; G01N 33/543; C01B 35/14; G01N 33/58

(52) U.S. Cl. CPC G01N 33/533; G01N 33/54346; B82Y 40/00; G01N 33/582; G01N 33/587; C01B 35/146

(57) ABSTRACT

An example compound according to an example of the present disclosure includes, among other possible things, a nanotube carrier, a moiety, a linker having first and second functional groups, wherein the first functional group is covalently linked to the nanotube carrier, and the second functional group is covalently linked to the moiety. An example method of making a nanotube compound according to the present disclosure is also disclosed.

### CROSS-REFERENCE TO RELATED APPLICATIONS

This application claims priority to U.S. Provisional Patent Application Ser. No. 62/855,128 filed May 31, 2019, which is hereby incorporated herein in its entirety. This application is a continuation-in-part of U.S. patent application Ser. No. 15/953,200, filed Apr. 13, 2018, which claims priority to U.S. Provisional Patent Application Ser. No. 62/485,379, filed Apr. 13, 2017, both of which are hereby incorporated herein in their entireties.

### STATEMENT REGARDING FEDERALLY-SPONSORED RESEARCH

The inventions described herein were made with government support under Grant #1261910, Grant #1521057 and Grant #1738466 awarded by the National Science Foundation. The Government has certain rights in this invention.

### BACKGROUND

Fluorophores are compounds with fluorescent properties that have biomedical applications. For example, fluorophores can be used as tracers or dyes for specific staining of certain molecules or structures. More particularly, fluorophores can be used to stain tissues, cells, or materials in a variety of analytical methods, such as fluorescent imaging and spectroscopy.

For the purpose of specific staining, fluorophores can be conjugated with biomolecules such as antibodies. However, reliable tracking and quantification of the fluorophores is challenging due to the low brightness and low photostability of commercial fluorophores. Therefore, a need exists for improved carrier molecules to carry fluorescent entities for biological and other applications. Other biological molecules may also benefit from improved carriers.

### SUMMARY

An example compound according to an example of the present disclosure includes, among other possible things, a nanotube carrier, a moiety, a linker having first and second functional groups, wherein the first functional group is covalently linked to the nanotube carrier, and the second functional group is covalently linked to the moiety.

An example method of making a nanotube compound according to the present disclosure includes, among other possible things, mechanically processing nanotubes in polar liquid, whereby the mechanical processing create imperfections on the nanotube and provides polar groups at the imperfections, and covalently linking a linker to the nanotubes, the linker having first and second functional groups, wherein the first functional group covalently links to the polar group.

### BRIEF DESCRIPTION OF THE DRAWINGS

FIG. 1 schematically shows compounds with nanomaterial carriers.

FIG. 2A shows an image of nanomaterial carriers of the compounds of FIG. 1.

FIG. 2B shows Fourier Transform Infrared Spectroscopy results for example nanomaterial carriers of the compounds of FIG. 1.

FIG. 3 shows excitation spectra between 245-270 nm of different batches of compounds with nanomaterial carriers.

FIG. 4 shows excitation spectra between 440-520 nm of different batches of compounds with nanomaterial carriers.

FIG. 5 shows XPS (x-ray photon spectroscopy) spectra of example compounds like those of FIG. 1 with an azide linker.

FIG. 6 shows fluorescence intensity for example compounds like those in FIG. 1 with an azide linker and FITC fluorescent entity.

FIG. 7A shows fluorescence intensity for example compounds like those in FIG. 1 with an azide-PEG linker and FITC fluorescent entity.

FIG. 7B shows fluorescence intensity for example compounds like those in FIG. 1 with an azide-PEG linker and sulforhodamine fluorescent entity.

FIG. 7C shows fluorescence intensity for example compounds like those in FIG. 1 with an azide-PEG linker and sulfoCy5.5 fluorescent entity.

FIG. 8A shows fluorescence intensity for example compounds like those in FIG. 1 with an amino linker and FITC fluorescent entity.

FIG. 8B shows fluorescence intensity for compounds with an amino linker and sulforhodamine fluorescent entity.

### DETAILED DESCRIPTION

Very generally, high-brightness fluorophores contain a carrier element, a fluorescent element, and a linker linking the carrier element to the fluorescent element. For biomedical applications, each of the carrier element, the linker, and the fluorescent element must be biocompatible (though the requirements for biocompatibility will vary with the particular application).

One example carrier element is a nanomaterial, such as carbon nanotubes (CNT) and boron nitride nanotubes (BNNTs), both of which can be used for biomedical applications such as cellular drug delivery and spectroscopy applications. However, it has been shown that fluorescent elements linked to certain nanotubes exhibit quenching, or a reduction in the brightness of the fluorescence.

It has been discovered that certain fluorophores having nanomaterial carriers not only do not exhibit the quenching effect, but also exhibit brightness several orders of magnitude higher than other known fluorophores, as will be discussed herein.

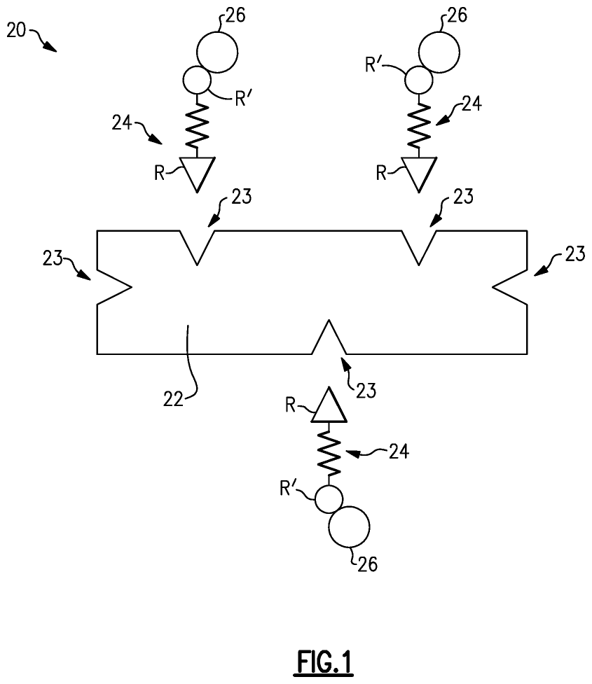

Referring now to FIG. 1, compounds 20 are shown. The compounds 20 generally comprise an inorganic nano-scale (“nanomaterial”) carrier 22, a linker 24, and a moiety 26. In some examples, the compound 20 includes more than one linker 24 and more than one moiety 26.

The carrier 22 is, in one example, a BNNT or CNT carrier. In a particular example, the carrier 22 is a multi-walled BNNT or CNT carrier, where each BNNT or CNT has multiple co-axial shells of hexagonal boron nitride (h-BN for BNNTs) or graphene (for CNTs), with a typical external diameter of more than about 0.4 nm but less than about 80 nm. The length of these BNNTs and CNTs is between about 100-2000 nm. In other examples, the carrier 22 can be another nano-scale inorganic material, such as hexagonal boron nitride (h-BN) nanosheets/nanoparticles and graphene/graphite nanosheets/nanoparticles, or zero-dimensional nanomaterials (“dots”).

The linker 24 has two or more functional groups R and R′, as shown in FIG. 1. The functional groups R and R′ are reactive groups that facilitate covalent bonding of the linker to other structures by any known chemistry. R and R′ can be the same or different type of functional group. The linker 24 can be any type of molecule that has two or more functional groups R and R′. One example linker 24 is a linear or branched polymeric molecule. In some examples, the linker 24 has a length of less than about 200 nm. In some examples, multiple linkers 24 can be connected to one another in series, e.g., a first linker 24 could be connected to a second linker 24 at the R′ group.

One functional group R interacts covalently with the carrier 22. A carrier 22 with a linker 24 is known as a “functionalized” carrier 22. That is, when covalently linked to linker 24, the carrier 22/linker 24 structure has a functional group R′ which facilitates covalent bonding of the carrier 22/linker 24 to another moiety 26. An example functional group R is a hydroxyl group and an example group R′ is an azide or amine group. However, R and R′ can be any known functional groups such as amine groups, carboxylic acid, isothiocyanate, maleimide, an alkyne group, an azide group, a thiol group, monosulfone, or an ester group such as a succinimidyl, sulfodichlorophenol, pentafluorophenyl or tetrafluorophenyl.

In some examples, multiple linkers 24 could be connected to one other in series. For instance, a second linker 24 could be connected to the functional group R′ of a first linker 24, the second linker 24 having its own R′ for covalent linking to the moiety 26.

The moiety 26 is, in one example, a fluorescent entity. In this example, the compound 20 is a fluorophore. The fluorescent entity is any fluorescent dye that is known in the art, including but not limited to coumarins, benzoxadiazoles, acridones, acridines, bisbenzimides, indole, benzoisoquinoline, naphthalene, anthracene, xanthene, pyrene, porphyrin, fluorescein, rhodamine, boron-dipyrromethene (BODIPY) and cyanine derivatives. Many such fluorescent dyes are commercially available. Fluorescent entities can also include tandem dyes which have two different dyes connected and which interact via FRET (fluorescence resonance energy transfer).

In other examples, the moiety 26 is a labelling moiety or other moiety to be delivered to a human body by the carrier 22, such as antibodies, peptides, DNAs, RNAs, oligonucleotides, or the like.

The moiety 26, in other examples, can be molecules and chelating agents with radioactive isotopes, ferromagnetic, and/or magnetic elements. In these examples, the compound 20 can be used as a contrast agent for medical imaging such as PET, SPECT, CT, MRI, etc.

In another example, the moiety 26 can include combinations of any of the example moieties 26 discussed above. In this example, the compound 20 can be used as a heterogeneous probe for biomedical detection and sensing.

Some nanomaterial carriers 22, and in particular, boron nitride-based nanomaterials, are known to be chemically inert. Therefore, it has been difficult to functionalize prior art nanomaterial carriers. However, it has been discovered that nanomaterial carriers that have been subject to mechanical processing in polar liquid (e.g., solution or solvent of surfactants) exhibit increased propensity to covalently interact with functional groups such as the functional group R on linker 24. Mechanical processing can be agitation, for instance. Furthermore, it has been discovered that mechanical processing of nanomaterial carriers improves the solubility of the nanomaterial carriers in aqueous solutions, which can be helpful for biocompatibility. Mechanical processing can also cut nanomaterial carriers 22 such as nanotubes to desired lengths after fabrication by any known method. Certain nanotube lengths, such as BNNTs with lengths between about between about 100 and 2000 nm, may have benefits in terms of biocompatibility, improved fluorescence when used as fluorophores, improved solubility in water, and/or other benefits, discussed in more detail below.

Nanotubes may be fabricated at lengths of between about 10,000-500,000 nm according to any known method and then cut to desired lengths. In a particular example, the nanotubes may be cut to lengths of between about 100 and 2000 nm by mechanical processing. However, longer nanotubes, or clumps of nanotubes may be used as carriers 22 as long as they can be functionalized and dispersed in biocompatible aqueous solution.

Accordingly, mechanical processing cuts nanotube carriers 22 (such as BNNTs or CNTs) to desired lengths, readies them for functionalization with linkers 24, and improves their solubility in aqueous solutions. One example method of mechanical processing is agitation. Agitation can be accomplished by sonication, such as tip sonication or bath sonication, for instance.

During mechanical processing in polar liquid (such as an aqueous solution of surfactant), imperfections 23 (FIG. 1) are formed in the nanotube carriers 22. Where the nanotube carriers 22 are tubes (for instance, BNNTs), imperfections 23 can be formed both at cutting edges of carriers 22, e.g., edges where the tubes are cut into shorter tubes during mechanical processing in solution, and along the lengths of the tubes 22. The imperfections 23 are disruptions or changes in the nanotube carrier 22 structure such that localized polarities or charges are exhibited at the imperfections 23. Polar or charged groups from the polar liquid interact with the localized polarities or charges at the imperfections. For example, for certain solutions, hydroxyl (—OH) groups from the solution may interact with the imperfections 23, though other solutions may have other polar or charged groups that can interact with the localized imperfections 23, such as amino, carboxylic acids, or aldehyde groups.

The polar or charged groups are themselves polar/charged and thus facilitate covalent interactions between the nanomaterial carrier 22 and the functional group R on linker 24. The polar/charged groups also increase the hydrophilicity of the nanotube carrier 22 by facilitating polar or ionic interactions with water molecules or ions in the water. Therefore, the nanotube carriers 22 exhibit improved solubility or dispersion in aqueous solution after mechanical processing in solution.

After mechanical processing, nanotube carriers 22 have multiple imperfections 23 and associated polar or ionic groups as discussed above. Each of these sites is available to interact covalently with functional group R of linker 24. Because there are multiple such sites on each nanomaterial carrier 22, multiple linkers 24 covalently interact with each nanomaterial carrier 22. Each linker 24 can covalently bond with a moiety 26, such as a fluorescent entity, via the functional group R′. Therefore, in the example where moieties 26 are fluorescent entities, the resulting fluorophore 20 has multiple fluorescent entities 26, the fluorescence of which are cumulative. The resulting fluorophore 20 thus has fluorescence that is orders of magnitude higher than prior art fluorophores. In other examples, as discussed in more detail below, moieties other than fluorescent entities 26 can be covalently bonded to the linker 24 via the functional group R′. In these examples, the nanomaterial carrier 22 can carry multiple moieties.

In one example method, BNNT carriers 22 are placed in polar liquid. The BNNT carriers 22 in solution then undergo mechanical processing in the form of agitation. In this example, the agitation is accomplished by sonication though a homogenizer or other method could also be used. The sonication results in cutting of the BNNT carriers 22. After sonication, the nanotube carriers 22 are washed to remove excess solution. For example, washing can be performed with deionized water in a centrifugal filter unit. In this example, the solution is a sodium cholate aqueous solution which results in hydroxylation of the BNNT carriers 22 (addition of hydroxyl functional groups to the carriers at imperfections 23), though other solutions could be used.

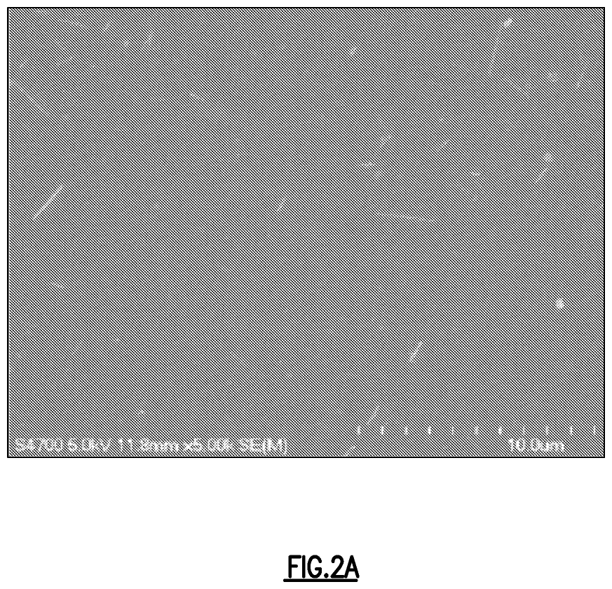

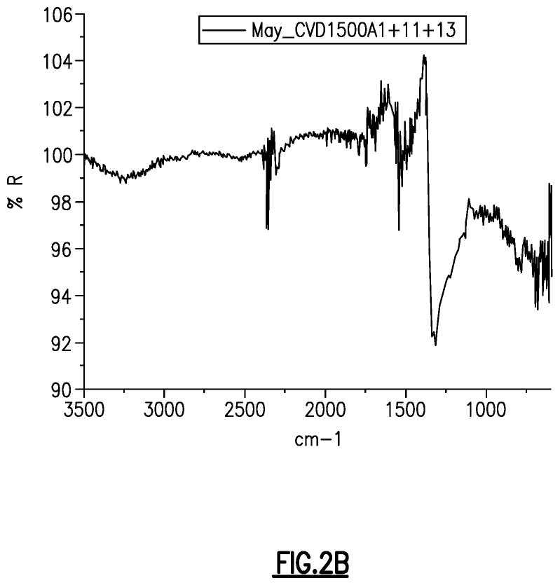

After sonication and washing, the BNNT carriers 22 have hydroxyl groups. This was confirmed by Fourier Transform Infrared Spectroscopy (FTIR) analysis, with results shown in FIG. 2B. In FIG. 2B, there is a peak between 3000-3500 cm⁻¹ which confirms the presence of a hydroxyl (—OH) group. Depending on the solution and functionalization process, other groups may be present after sonication and washing.

Furthermore, the BNNT carriers 22 that underwent agitation in surfactant solution exhibit improved solubility in water after the agitation. It has also been discovered that solubility varies inversely with BNNT carrier 22 length. That is, shorter BNNT carriers 22, e.g., BNNT carriers with lengths between about 100 and 350 nm, exhibit better dispersion (e.g., solubility) than longer BNNT carriers 22, e.g., BNNT carriers with lengths between about 500 nm and 2000 nm.

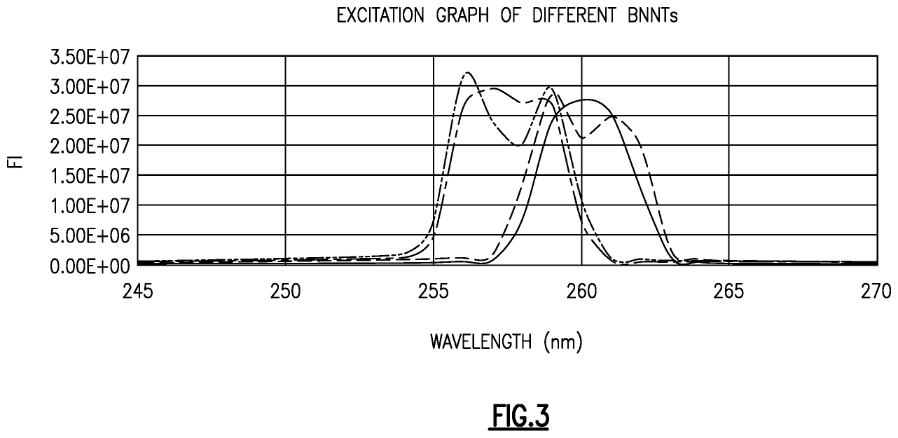

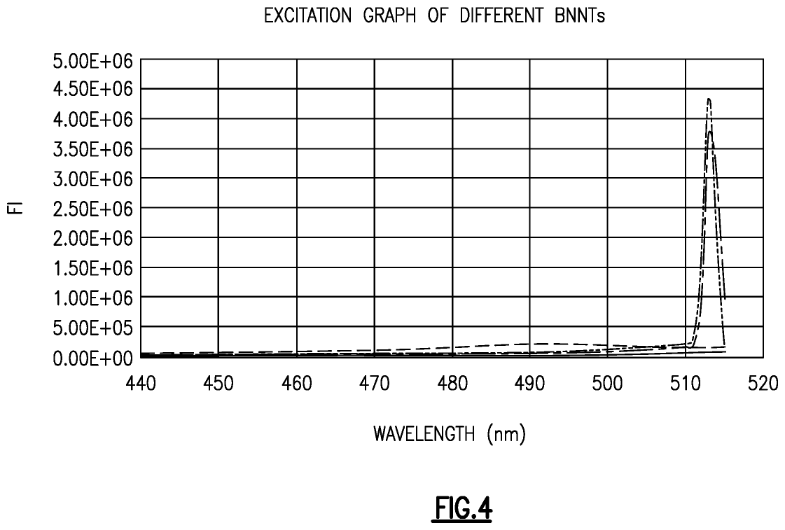

It has also been discovered that BNNT carriers 22 that underwent tip sonication exhibit autofluorescence (that is, the BNNT carrier 22 themselves fluoresce, without being conjugated with a fluorescent entity 26). FIGS. 3-4 show excitation graphs for several example batches of BNNT carriers 22 that underwent agitation as described above. The functionalized BNNT carriers 22 formed by the method described above fluoresce at about 250-265 nm with a fluorescence intensity of between about 2×10⁷ and 3×10⁷ and at about 510-520 nm with a fluorescence intensity of between about 3.5×10⁶ and 4.5×10⁶, as shown in FIGS. 3-4. Uncut BNNT carriers that did not undergo mechanical processing do not fluoresce at all.

In one particular example, the linker 24 is an azide linker. An example azide linker 24 is 3-azidopropyl-triethyoxysilane, though other azide linkers 24 are also contemplated. 3-azidopropyl-triethyoxysilane can be added to the nanomaterial carrier 22 as shown in Equation 1 below after the nanomaterial carrier 22 undergoes mechanical processing:

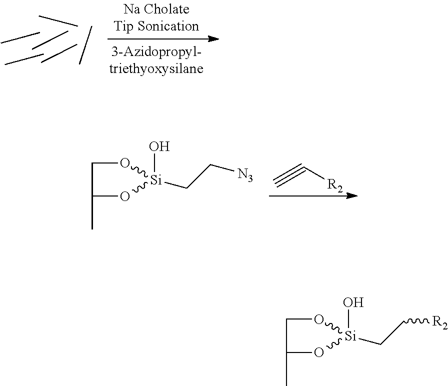

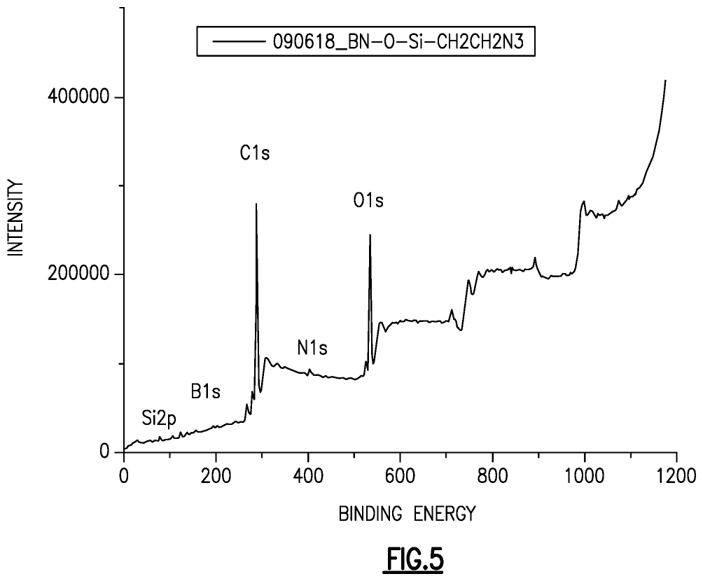

As shown in Equation 1, hydroxyl groups on the BNNT carrier 22 covalently bond to the silicon atom of the azide linker 24 to form an intermediate carrier 22/linker 24 (however, in other examples, the functionalized carrier 22 may have different functional groups available for covalent bonding, as discussed above). The azide (N₃) group in this example is the functional group R′ of the linker 24. Then, the intermediate carrier 22/linker 24 is covalently joined to a moiety R2 by any known chemistry. FIG. 5 shows XPS (x-ray photon spectroscopy) spectra of functionalized carriers 22 with 3-azidopropyl-triethyoxysilane linkers 24, and confirms successful covalent functionalization with the BNNT carriers 22 with the expected peaks.

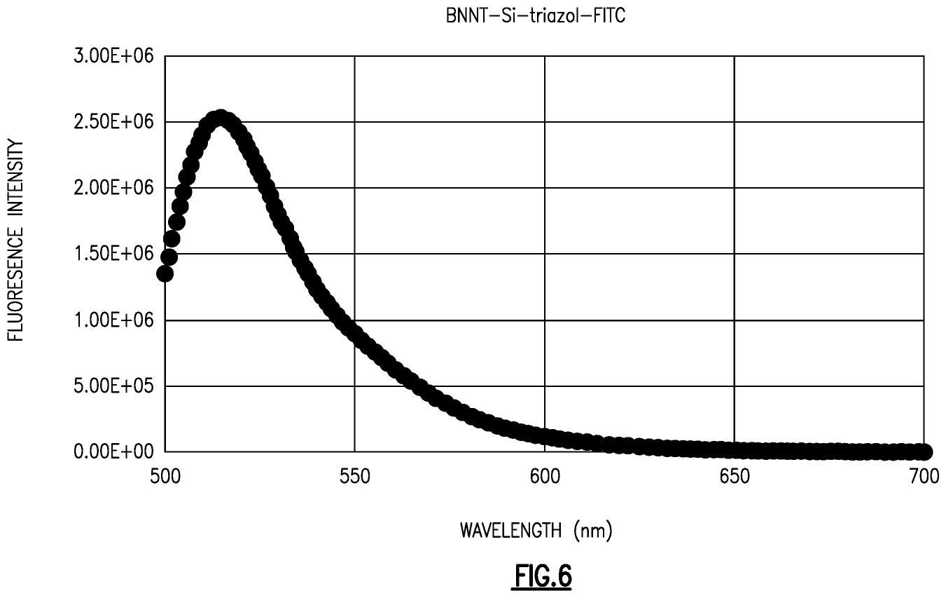

In a particular example, the moiety R2 in Equation 1 is FITC, a green fluorescent entity 26, and the resulting compound is a green fluorophore 20. Due to the mechanical processing of the nanomaterial carrier 22 and the availability of functional groups for covalent bonding to the linker 24, the functionalized carrier 22 bonds to more linkers 24 and thus more fluorescent entities 26 than prior art nanomaterial carriers, as discussed above. Accordingly, the resulting green fluorophore 20 exhibits fluorescence intensity several orders of magnitude higher than prior art fluorophores when comparing at the same concentration of fluorophore unit. At about 520 nm, the green fluorophore 20 has a fluorescence intensity of about 2.5×10⁶, as shown in FIG. 6. These samples have about 108 to 1012 fluorophores 20.

In another particular example, the nanomaterial carrier 22/azide linker 24 was further functionalized with a second linker 24 that contains a PEG (polyethylene glycol) chain, such as alkyne-PEGSK-amino, wherein the amino (NH₂) group is a functional group R′, according to Equation 2 below. The PEG group improves solubility of the resulting carrier 22/linker 24 in water, so that it can be more easily dispersed in aqueous solutions like PBS, plasma, etc. Solubility in water may be desirable for certain biological applications. The length, composition, and degree of branching of the PEG chain can be selected to obtain the desired solubility. Furthermore, it should be understood that other organic chains could be used instead of PEG to the same effect. The intermediate carrier 22/linker 24 is covalently joined to a moiety R2 by any known chemistry.

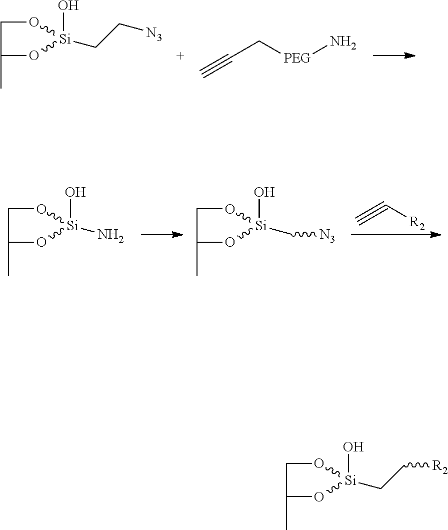

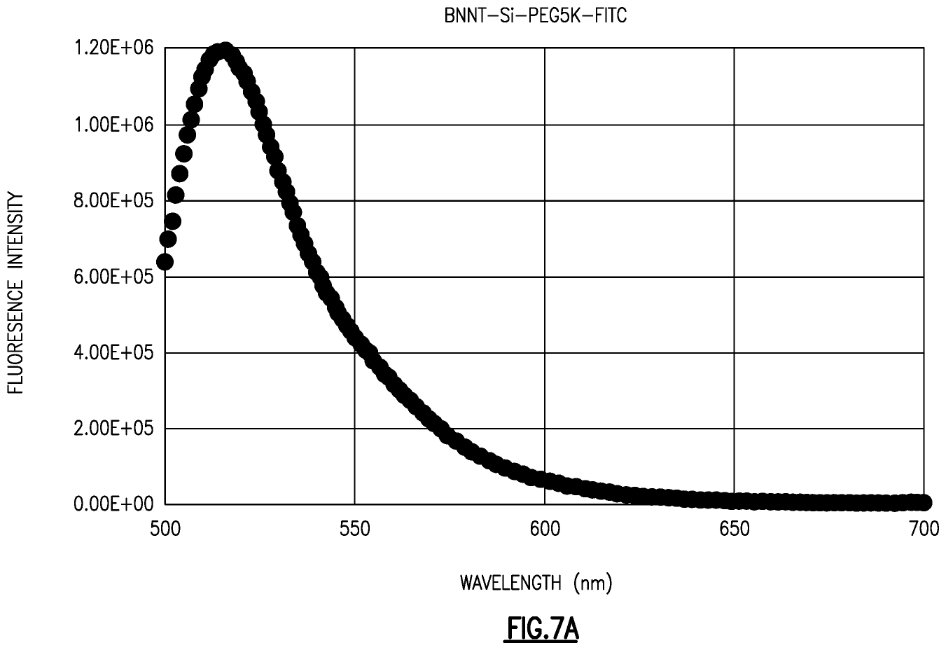

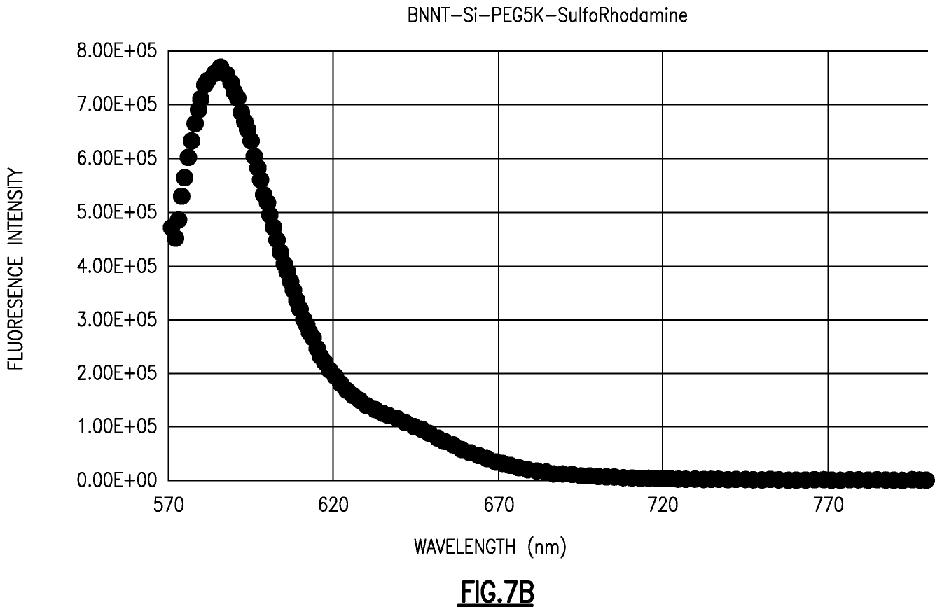

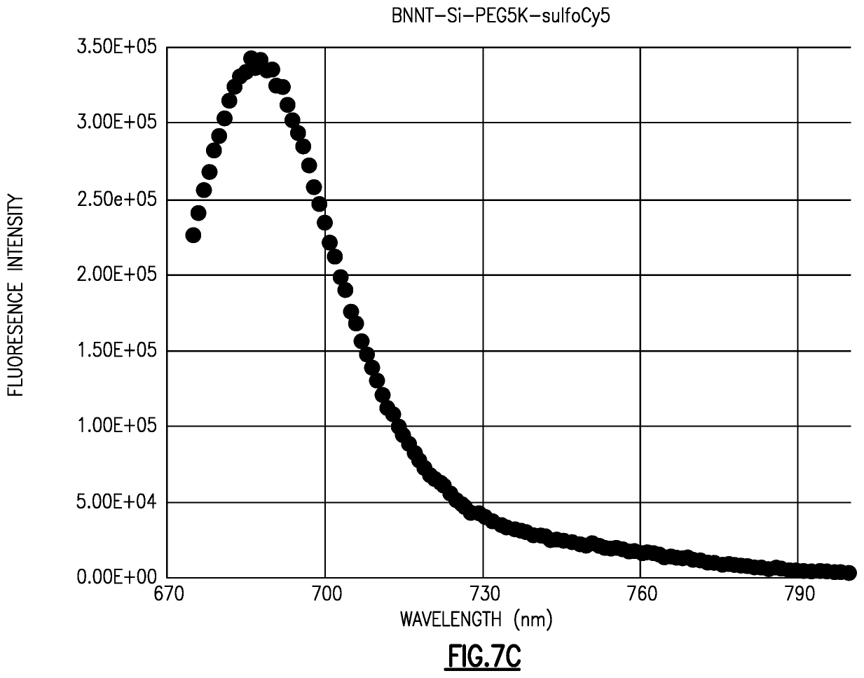

FIGS. 7A-C, respectively, show fluorescence intensity for fluorophores 20 made by adding FITC (green), sulforhodamine (red), and sulfocy5.5 (far-red) fluorescent entities 26 to the carrier 22/linker 24 shown in Equation 2, whereby the fluorescent entities 26 are covalently linked to the linker 24 functional group by any known chemistry. As shown, the green fluorophore 20 has a fluorescence intensity of about 1.2×10⁶ at 520 nm, the red fluorophore has a fluorescence intensity of about 7.8×10⁶ at about 580 nm, and the far-red fluorophore has a fluorescence intensity of about 3.5×10⁵ at about 685 nm. These samples have about 108 to 1012 fluorophores 20 and the fluorescence intensity per fluorophore 20 is hundreds to thousands of times higher than those of prior art fluorophores due to different number of fluorescent entities 26 loading by covalent bonding.

The length of the PEG or other organic chain in the example discussed above may in some cases affect the brightness of the resulting fluorophore 20. For instance, fluorophores 20 without a PEG or organic chain prepared as shown in Equation 1 above (“direct bonding fluorophores”) are less bright than fluorophores with a PEG or organic chain prepared as shown in Equation 2 above (“chain fluorophores”) after adjusting for sample size (e.g., number of fluorophores 20 in the samples used for measuring fluorescence intensity as shown in FIGS. 6 and 7A, respectively). It was observed that the direct bonding fluorophores prepared as shown in Equation 1 exhibit only two times higher fluorescence intensity that the chain fluorophores prepared as shown in Equation 2 above, even though the sample of direct bonding fluorophores had 571 times the concentration of fluorophores than the sample of chain fluorophores. This suggests that the use of longer linker 24 (e.g., a linker 24 with a PEG or other organic chain) could enhance the solubility and brightness of the fluorophores 20. Without being bound by any particular theory, this could be due to reduction of the quenching effect discussed above.

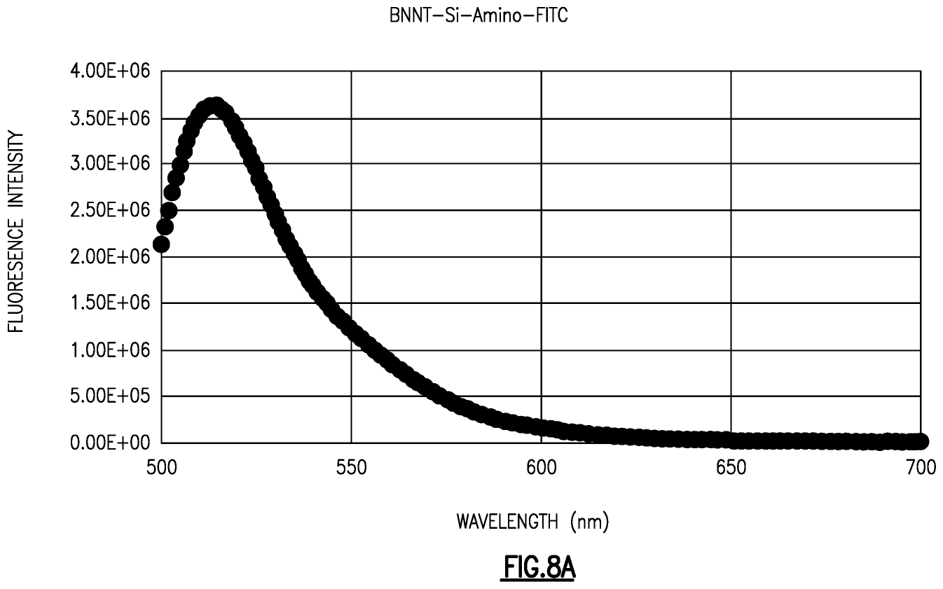

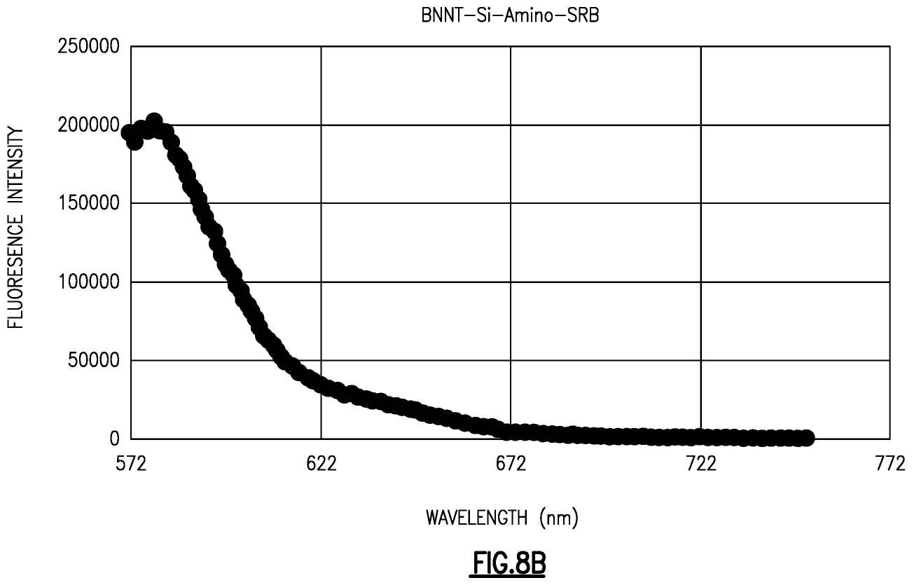

In another particular example, an amine linker 24 is added to the nanomaterial carrier 22. An example amine linker is 3-aminopropyltrimethoxysilane. As above, the silicon atom of 3-aminopropyltrimethoxysilane covalently bonds to the functional groups (for instance, hydroxyl groups) on the functionalized carrier 22 to form an intermediate carrier 22/linker 24. FIGS. 8A-B, respectively, show fluorescence intensity for fluorophores 20 made by adding FITC (green) and sulfrorhodamine (red) fluorescent entities 26 to the carrier 22/linker 24 which resulted in covalently linking the fluorescent entities 26 to the functional group R′ of the linker 24, which in this example is the amine group. As shown, the green fluorophore 20 has a fluorescence intensity of about 3.7×10⁶ at 520 nm and the red fluorophore has a fluorescence intensity of about 2×10⁵ at about 580 nm. These samples (fluorophores 20) have about 108 to 1012 fluorescent entities 26 due to different dye loading.

In addition to the azide and amine example linkers 24 discussed above, other linkers with other functional groups could be used. For instance, any known functionalization practice could be followed using a linker 24 including alkyne-NHS, when can then be covalently bonded to amino groups at the Fc groups of an antibody. Other small molecules (sugars, nitroxides, biotin, drugs, etc.), macromolecules, peptides, DNA, RNA sequences, proteins such as biotin, SA (streptavidin and its derivatives) could be used as moiety 26 for targeting.

Furthermore, linking of the functionalized carriers 22 are not restricted with amino-silanes linkers 24. Other linkers 24 might have a variety of functional groups such as amino, carboxylic acid, succinimdyl ester, maleimide, carboimide, pyridyldithiol, haloacetyl, aryl azide, azide, alkyne, hydrazide and monosulfone groups. Those groups could be used for the conjugation of carriers 22 to dye, drug, or any targeting material. Cross-linkers which contain dual functional group can also be used to obtain functional group to conjugate linkers 24 to other entities such as dye, peptide, oligonucleotide, DNA, RNA, antibody, proteins, drugs or other nanoparticles. Those cross-linkers might be SMCC (succinimidyl 4-(N-maleimidomethyl)cyclohexane-1-carboxylate), sulfo-SMCC ((sulfo-succinimidyl 4-(N-maleimidomethyl)cyclohexane-1-carboxylate), AMAS (N-α-maleimidoacet-oxysuccinimide ester), BMPS (N-β-maleimidopropyl-oxysuccinimide ester), GMBS (N-γ-maleimidobutyryl-oxysuccinimide ester), sulfo-GMBS, MB S (m-maleimidobenzoyl-N-hydroxysuccinimide ester), sulfo-MBS, EMCS (N-ε-malemidocaproyl-oxysuccinimide ester), sulfo-EMCS, SMPB (succinimidyl 4-(p-maleimidophenyl)butyrate), sulfo-SMPB, SMPH (Succinimidyl 6-((beta-maleimidopropionamido)hexanoate), LC-SMCC succinimidyl 4-(N-maleimidomethyl)cyclohexane-1-carboxy-(6-amidocaproate), sulfo-KMUS (N-κ-maleimidoundecanoyl-oxysulfosuccinimide ester), SM(PEG)n where n=2,4,6,8,12,24 (PEGylated SMCC cross-linker), SPDP (succinimidyl 3-(2-pyridyldithio)propionate), LC-SPDP, sulfo-LC-SPDP, SMPT (4-succinimidyloxycarbonyl-alpha-methyl-α(2-pyridyldithio)toluene), PEGn-SPDP (where n=2,4,12,24), SIA (succinimidyl iodoacetate), SBAP (succinimidyl 3-(bromoacetamido)propionate), SIAP (succinimidyl (4-iodoacetyl)aminobenzoate), sulfo-SIAP, ANB-NOS (N-5-azido-2-nitrobenzoyloxysuccinimide),sulfo-SANPAH (sulfosuccinimidyl 6-(4′-azido-2′-nitrophenylamino)hexanoate), SDA (succinimidyl 4,4′-azipentanoate), sulfo-SDA, LC-SDA, sulfo-LC-SDA, SDAD (succinimidyl 2-((4,4′-azipentanamido)ethyl)-1,3′-dithiopropionate), Sulfo-SDAD, DCC (N,N′-Dicyclohexylcarbodiimide), EMCH (N-ε-maleimidocaproic acid hydrazide), MPBH (4-(4-N-maleimidophenyl)butyric acid hydrazide), KMUH (N-κ-maleimidoundecanoic acid hydrazide), PDPH (3-(2-pyridyldithio)propionyl hydrazide), PMPI (p-maleimidophenyl isocyanate), SPB (succinimidyl44-(psoralen-8-yloxy)l-butyrate), or other known cross-linkers.

Though the preceding description is made with respect to BNNTs, CNTs can be functionalized and act as carriers 22 as well. CNTs can be functionalized by agitation, as discussed above with respect to BNNTs. CNTs are also known in the art to be responsive to functionalization by chemical means, such as acid treatment. Like functionalized BNNT carriers 22, functionalized CNT carriers 22 exhibit autofluorescence and exhibit improved capacity to link to fluorescent entities. Accordingly, fluorophores 20 with CNT carriers 22 exhibit increased fluorescence as compared to prior art fluorophores with non-functionalized carriers.

### EXAMPLES

Synthesis of BNNT-Si-azide:Sodium cholate cut BNNTs were excessively washed by using 10 KDa at 4000 g for 5 min with distilled water about 5-6×. This process also results in concentrated BNNT sample. Later, metal contamination was cleaned with acid treatment, with HCl or HNO3. After acid treatment, BNNTs were neutralized and excessively washed with distilled water during vacuum filtration from anodic membranes (pore size: 20 nm). Collected BNNT-OH were resuspended in EtOH using sonication bath. Later, ethanol was evaporated and BNNTs (2.5 mg) were suspended inside 5 ml toluene, and 1 ml ethanol. Then, 20 ul (3-azidopropyl)triethoxysilane was added and stirred overnight, under nitrogen at 110 degrees C. The next day, solvent was evaporated and resuspended in EtOH and BNNTs were washed and collected on anodic membranes (pore size: 20 nm) via vacuum filtration. BNNTs were analyzed with XPS to confirm the presence of Si.

Synthesis of BNNT-Si-triazol-FITC:BNNT-Si-azide (500 ul) were mixed with 20 ul of sodium ascorbate (0.12M) and 20 ul of copper sulfate (0.12M). Then, 2 ul FITC alkyne (3190 nM) from stock solution in EtOH/DMSO was added. After stirring overnight, BNNTs were washed with distilled water, and excessively with EtOH (70%) through anodic membranes via vacuum filtration. Later, it was resuspended in EtOH to collect BNNTs into glass vial. Solvent was evaporated and dried overnight. Later, BNNT-Si-FITC was resuspended inside 1 ml PBS and fluorescence intensity was measured via Horiba Fluoromax-4.

Synthesis of BNNT-Si-amine-BNNT-OH (1 mg) was dispersed in 5 mL toluene and 50 μl (3-Aminopropyl)triethoxysilane (APTES) was added. The reaction was heated to reflux and stirred under nitrogen overnight. Then the solvent was removed through evaporation under reduced vacuum. The residue was dispersed in 70% ethanol and was washed through anodic membranes with 70% ethanol and water. The precipitant was collected through rinsing membranes with ethanol and concentrating solvent. Then the product was dispersed in 2 mL anhydrous DMSO and kept 4° C. under nitrogen.

Synthesis of BNNT-Si-amine sulforhodamine:BNNT-silicone amine (500 ul in DMSO) was withdrawn from stock solution and mixed with 5 ul sulforhodamine acid B chloride (15 mM in DMSO), one drop triethylamine and 1 ml chloroform. The reaction was proceeding under nitrogen overnight. The chloroform was removed through evaporation and then the residue was diluted in 5 ml water and kept DMSO less 10%. The product was collected through filtration from anodic membranes.

Synthesis of BNNT-Si-amine FITC: The procedure is same as for the sulforhodamine. Fluorescein isothiocyanate (3.2 mM in EtOH+DMSO) was used as starting material.

Synthesis of BNNT-Si-triazol-PEGSK-azide:BNNT-Si—N3 (0.3 mg in 1 ml distilled water) was mixed with 20 ul CuSO₄ (0.12M) and 20 ul sodium ascorbate (0.12M). Then, 5.3 mg alkyne-PEG5K-amino was added. The mixture was stirred at room temperature for 2 h, then stirred overnight then extracted with chloroform. The mixture was then evaporated and dispersed in saturated sodium bicarbonate, and 45 mg CuSO₄ was added. Then stock solution of triflic azide was added. Triflic azide was synthesized according to known protocol. Sodium azide (0.4 g) was dissolved in 1 ml distilled water and 1 ml toluene, in ice bath. Then, 0.6 g triflic anhydride was added drop by drop and stirred for 2 h. Then, extracted with 6 ml toluene. This stock solution was used to convert amino functional group into azide. After addition of triflic azide into BNNT-Si-triazol-PEG5K-azide, it was stirred overnight and BNNTs were extracted with chloroform. After evaporation, they were dispersed in PBS. Transmittance was measured in order to determine the concentration.

Synthesis of BNNT-Si-triazol-PEG5K-triazol-dye:BNNT-Si-triazol-PEG5K-azide (500 ul, at a concentration of 83 ug/ml) was already dispersed in PBS. CuSO₄ (20 ul, 0.12M) and sodium ascorbate (20 ul, 0.12M) were added into BNNT-azide, then 20 ul of FITC-alkyne (3.2 mM) was added and stirred overnight in the fridge. The next day, it was filtered through anodic membranes, washed excessively with water, and 70% EtOH. Later, it was dispersed in PBS and fluorescence were measured on Horiba Fluoromax-4. Similarly, in the case of sulforhodamine, we used 20 ul sulforhodamine alkyne (15 mM) and for sulfocyanine dye synthesis we used 20 ul of sulfocyanine 5 alkyne (3.5 mM, dissolved in DMSO:Water (1:1)). Excess dye were removed by excessive washing with distilled water and 70% EtOH.

Synthesis of antibody alkyne:Anti-human CD4 (200 ug) was washed three times with PBS to remove sodium azide and concentrate the sample by using 30 KDa filter unit down to 80 ul. Then, 20 ul DTT was added from 100 mM stock solution, dispersed in distilled water. After 30 mM, the antibody was washed with PBS about 5× to remove DTT. After concentrating reduced antibody, it was mixed with 100 ul maleimide-PEG4-alkyne (10.2 mg/ml in PBS). It was then stirred overnight and purified by using 30 KDa filter unit. Product formation was confirmed by SDS non reducing gel at 150V for 70 min.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

What is claimed is:

1. A compound, comprising:
a nanotube carrier;
a moiety; and
a linker having first and second functional groups, wherein the first functional group is covalently linked to the nanotube carrier, and the second functional group is covalently linked to the moiety.

2. The compound of claim 1, wherein the nanotube carrier is a boron nitride nanotube (BNNT).

3. The compound of claim 2, wherein the boron nitride nanotube has a length between about 100 and 2000 nm.

4. The compound of claim 1, wherein the nanotube carrier is a carbon nanotube (CNT).

5. The compound of claim 1, wherein the nanotube carrier is a multi-walled nanotube carrier.

6. The compound of claim 1, further comprising a plurality of linkers covalently linked to the nanotube carrier, and a plurality of moieties, wherein each linker is linked to a moiety of the plurality of moieties.

7. The compound of claim 1, wherein the nanotube carrier has at least one polar group, and wherein the first functional group is covalently linked to the nanotube carrier at the at least one polar group.

8. The compound of claim 7, wherein the at least one polar group is a hydroxyl (—OH) group.

9. The compound of claim 1, wherein the moiety includes at least one of one of a fluorescent entity, a biological molecule, a chelating agent, and combinations thereof.

10. The compound of claim 1, wherein the linker is a first linker, and further comprising a second linker having third and fourth functional groups, wherein the second linker is covalently linked to the first linker via covalent interaction between the second and third functional groups, and the moiety is covalently linked to the fourth functional group.

11. A method of making a nanotube compound, comprising:
mechanically processing nanotubes in polar liquid, whereby the mechanical processing create imperfections on the nanotube and provides polar groups at the imperfections; and
covalently linking a linker to the nanotubes, the linker having first and second functional groups, wherein the first functional group covalently links to the polar group.

12. The method of claim 11, wherein the mechanical processing results in cutting the nanotubes.

13. The method of claim 12, wherein the nanotubes have lengths between about 100 and 2000 nm after the mechanical processing.

14. The method of claim 11, wherein the moiety is a fluorescent entity.

15. The method of claim 11, wherein the mechanically processing includes agitation.

16. The method of claim 15, wherein the agitation is accomplished by sonication or by homogenizer.

17. The method of claim 11, wherein the polar groups are hydroxyl (—OH) groups.

18. The method of claim 11, wherein the nanotubes are boron nitride nanotubes (BNNTs).

19. The method of claim 11, wherein the nanotubes are carbon nanotubes (CNT).

20. The method of claim 11, wherein the nanotubes are multi-walled nanotubes.
